(19) United States
(12) Patent Application Publication
(10) Pub. No.: US 2012/0116148 A1
(43) Pub. Date: May 10, 2012

(54) MAGNETIC-ASSISTED TUMOR CONFINEMENT METHODOLOGY AND EQUIPMENT

(75) Inventors: Irving N. WEINBERG, Bethesda, MD (US); Benjamin SHAPIRO, College Park, MD (US)

(73) Assignee: WEINBERG MEDICAL PHYSICS LLC, Bethesda, MD (US)

(21) Appl. No.: 13/290,751

(22) Filed: Nov. 7, 2011

Related U.S. Application Data
(60) Provisional application No. 61/411,200, filed on Nov. 8, 2010.

(51) Int. Cl. A61N 2/00

(52) U.S. Cl. 600/12

(57) ABSTRACT

Disclosed embodiments are directed to reducing the likelihood of intra-operative shedding. Specifically, disclosed embodiments utilize injected magnetic nanoparticles and magnets to confine tumor cells to the surgical bed in order to prevent the cells from being released into the general circulation and/or lymphatics.

### CROSS-REFERENCE TO RELATED APPLICATION(S)

This is application relies for priority on U.S. Provisional Patent Application Ser. No. 61/411,200, filed on Nov. 8, 2010, the entirety of which being incorporated by reference herein.

### FIELD OF THE INVENTION

Disclosed embodiments are directed, generally, to medical treatment of tumors.

### DESCRIPTION OF THE RELATED ART

Many victims of cancer die of disseminated metastases, rather than from local growth of the cancer. Jemal A, et al. Cancer statistics. CA Cancer J Clin. 58(2):71-96 (2008).

Metastases are the result of Disseminated Tumor Cells (“DTC”), which are constructs that encompass Circulating Tumor Cells (CTCs), micro-metastases, and lymphatic tumor infiltration.

CTCs were first observed in cancer patients over a century ago. The role of CTCs in causing metastases is a source of speculation and somewhat controversial. High numbers of CTCs correlate with reduced survival in patients. R Schuster, N E Bechrakis, A Stroux, A Busse, A Schmittel, C Scheibenbogen, E Thiel, M H Foerster, U Keiholz. Circulating Tumor Cells as Prognostic Factor for Distant Metastases and Survival in Patients with Primary Uveal Melanoma. Clin Cancer Res 13(4):1171-1178 (2007). However, some cancer biologists surmise this may be due to active proliferation of existing metastases, rather than primary tumor shedding that may cause subsequent metastatic activity. J Eyles, A-L Puaux, X Wang, B Toh, C Prakash, M Hong, T G Tan, L Zheng, L C Ong, Y Jin, M Kato, A Prevost-Blondel, P Chow, H Yang, J-P Abastado. Tumor cells disseminate early, but immunosurveillance limits metastatic outgrowth, in a mouse model of melanoma. J Clin Invest 120(6):2030-2039 (2010).

Despite differences of opinion as to the role of CTCs in spreading cancer, clinical trials have established an association between tumor relapse and the detection of CTCs during and after cancer surgery. J Weitz et al. Dissemination of Tumor Cells in Patients Undergoing Surgery for Colorectal Cancer. Clinical Cancer Research 4:343-348 (1998).

In contrast, no association has been found between relapse and preoperative CTC levels. M Koch et al. Detection of Hematogenous Tumor Cell Dissemination Predicts Tumor Relapse in Patients Undergoing Surgical Resection of Colorectal Liver Metastases. Ann Surg 241:199-205 (2005).

Recent methodological improvements have shown that large numbers of CTCs are released during cancer surgery. N Sawabata et al. Circulating tumor cells in peripheral blood caused by surgical manipulation of non-small-cell lung cancer: pilot study using an immunocytology method. Gen Thorac Cardiovasc Surg 55:189-192 (2007). Ratios of pre-surgical to post-surgical CTC assays have been determined as high as 315:1. P Papavasiliou et al. Circulating tumor cells in patients undergoing surgery for hepatic metastases from colorectal cancer. Proc Baylor University Medical Center 23(1):11-14 (2010).

This observation has led to investigation of medical strategies for reducing the potential for iatrogenic (i.e., caused or aggravated by medical treatment) spread of metastasis. See, B Taylor. Detection of Hematogenous Tumor Cell Dissemination Predicts Tumor Relapse in Patients Undergoing Surgical Resection of Colorectal Liver Metastases. Editorial. Ann Surg 241:206-207 (2005) and also Abdalla E K, Vauthey J N, Ellis L M, Ellis V, Pollock R, Broglio K R, Hess K, Curley S A. Recurrence and outcomes following hepatic resection, radiofrequency ablation, and combined resection/ablation for colorectal liver metastases. Ann Surg 2004; 239(6): 818-825.

Approaches to hepatic resection that avoid liver manipulation before venous occlusion have also demonstrated improved survival. Liu C L, Fan S T, Lo C M, et al. Anterior approach for major right hepatic resection for large hepatocellular carcinoma. Ann Surg. 2000; 232:25-31. To quote an editorial in the 2005 Annals of Surgery: “Perhaps the most sobering message in this paper is the suggestion of intraoperative shedding of cells, which may directly implicate the surgeon in upstaging the disease”.

### SUMMARY

The following presents a simplified summary in order to provide a basic understanding of some aspects of various invention embodiments. The summary is not an extensive overview of the invention. It is neither intended to identify key or critical elements of the invention nor to delineate the scope of the invention. The following summary merely presents some concepts of the invention in a simplified form as a prelude to the more detailed description below.

Disclosed embodiments are directed to reducing the likelihood of intra-operative shedding. Specifically, disclosed embodiments utilize injected magnetic nanoparticles and magnets to confine tumor cells to the surgical bed in order to prevent the cells from being released into the general circulation and/or lymphatics.

### BRIEF DESCRIPTION OF THE DRAWINGS

A more compete understanding of the present invention and the utility thereof may be acquired by referring to the following description in consideration of the accompanying drawings, in which like reference numbers indicate like features, and wherein:

FIGS. 1A-D illustrate the provision of magnetic nanoparticles for use in accordance with the disclosed embodiments.

FIGURES. 2A-B illustrate results of preliminary studies of magnetic confinement in which, MDA-MB-231 cells were cultured onto sixteen cover slips and allowed to grow to confluency.

FIG. 3 illustrates the quantitative analysis of the images to determine the amount of cells that remained as a result of the exposure to the nanoparticles and the magnetic field.

### DETAILED DESCRIPTION

The description of specific embodiments is not intended to be limiting of the present invention. To the contrary, those skilled in the art should appreciate that there are numerous variations and equivalents that may be employed without departing from the scope of the present invention. Those equivalents and variations are intended to be encompassed by the present invention.

In the following description of various invention embodiments, reference is made to the accompanying drawings, which form a part hereof, and in which is shown, by way of illustration, various embodiments in which the invention may be practiced. It is to be understood that other embodiments may be utilized and structural and functional modifications may be made without departing from the scope and spirit of the present invention.

Moreover, it should be understood that various connections are set forth between elements in the following description; however, these connections in general, and, unless otherwise specified, may be either direct or indirect, either permanent or transitory, and either dedicated or shared, and that this specification is not intended to be limiting in this respect.

Disclosed embodiments are directed to reducing the likelihood of intra-operative shedding. Specifically, disclosed embodiments utilize injected magnetic nanoparticles and magnets to confine tumor cells to the surgical bed in order to prevent the cells from being released into the general circulation and/or lymphatics.

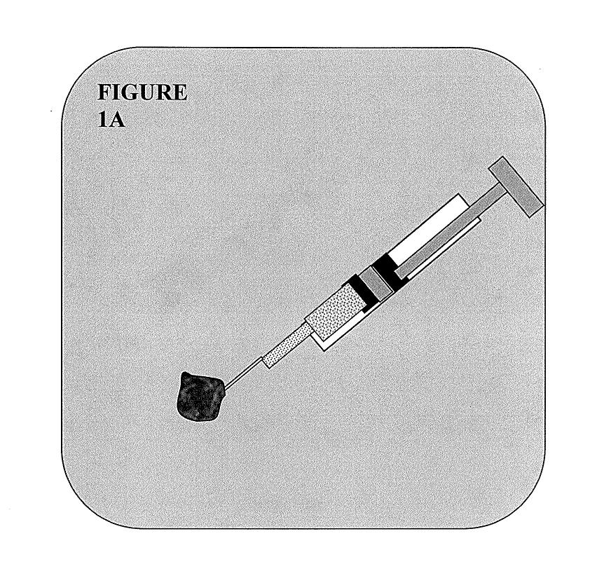

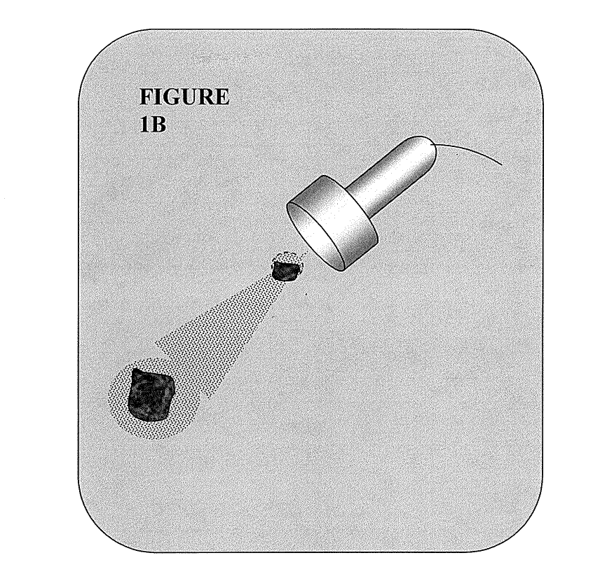

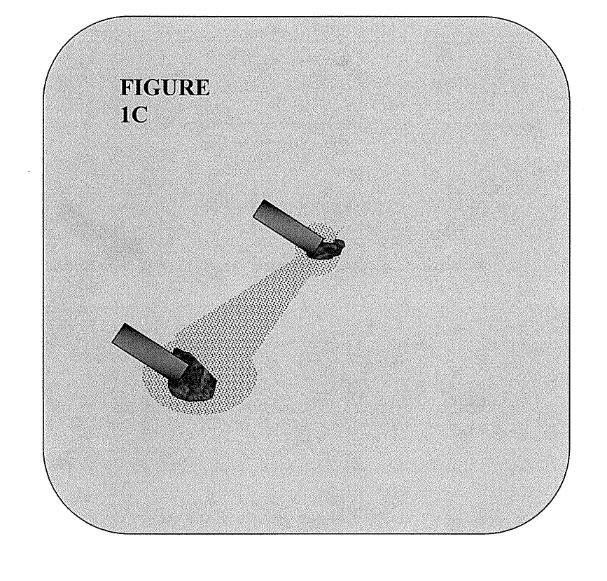

FIGS. 1A-1D illustrate the provision of magnetic nanoparticles for use in accordance with the disclosed embodiments. As part of such use, the source of applied magnetic fields may be, for example, permanent-magnet surgical clips, as illustrated in FIGS. 1A-1D. However, it should be appreciated that alternative configurations for the source of applied magnetic fields could include implantable magnetic seeds, probes, or retractors to attract tumor cells.

The main application of the confinement concept may be to assist patients undergoing curative surgery. In addition to this main application, confinement of cells may also be a useful strategy in patients who (for various reasons) are poor candidates for curative surgical tumor resection. Clinical data shows continued shedding of tumor cells occurs in patients with unresected gastric cancers. S Matsusaka et al. Circulating tumor cells as a surrogate marker for determining response to chemotherapy in patients with advanced gastric cancer. Cancer Science 101(4):1067-1071 (2010).

A patient who will undergo surgery would be treated with magnetic elements. For example, magnetic micro or nanoparticles, would be injected by a hypodermic needle into the tumor. The magnetic elements could also be magnetic micro or nano-rods, polymer capsules, micelles or liposomes with magnetic materials, animal or human cells loaded with magnetic particles or other magnetic materials, or they could be magnetic particles of other shapes. The magnetic elements could be made out of magnetically responsive materials, e.g., ferromagnetic, ferrimagnetic, paramagnetic, or diamagnetic materials, or such materials could be located inside the elements or affixed or attached to their external surface. Alternatively, magnetic elements could be administered to the patient systemically, by ingestion, topically, or by intravenous fluid application.

The magnetic elements could be selected (e.g., functionalized, shaped, chemically or genetically modified) so that they are more likely to enter, bind with, attach to, be endocytosed by, or otherwise be more likely to be associated with the patients tumor cells than with other cells.

Once the magnetic elements are associated with a tumor or tumor cells, during surgery, a permanent or electromagnet could be applied to the tumor or location of surgery to magnetically confine tumor cells to a region by magnetic action on the magnetic elements now associated with the tumor cells. Thus, application of the magnetic field would reduce the risk of shedding tumor cells during surgery.

For example, magnetic forces could be used to confine tumor cells to the tumor, or they could be used to confine tumor cells to the vicinity of the tumor, or to the surgical bed. After the surgeon has completed actions that could cause tumor cells to be shed (e.g. after the surgeon has completed resecting the tumor), magnetic forces could be used to collect shed tumor cells attached or now containing magnetic elements. The magnets could then be removed taking with them shed tumor cells to decrease the number of tumor cells that would otherwise be shed into the body of the patient.

Further, magnetic elements and magnetic forces could be used to isolate tumor cells from the circulation or lymphatics of the patient. Magnetic imaging could also be used to monitor the distribution of shed tumor cells, and could thus be used to check if any tumor cells have reached sentinel nodes. Magnetic forces could also be used to remove shed tumor cells from sentinel nodes, a medically desirable outcome. The location of sentinel nodes could be detected by means of a magnetometer, or by ultrasound assistance. Magnets to apply magnetic forces to the magnetic elements could be positioned at needed locations by implanting a magnet into the patient, or by having a permanent or electromagnet held in place by an outside support. Magnetic fields and forces could be applied prior to, during, and after surgery.

Many patients with prostate cancer choose to have their tumors treated non-surgically. There is a clear association with the presence of CTC and prostate cancer. (See, P O Ts'o et al. Detection of intact prostate cancer cells in the blood of men with prostate cancer, Urology 49(6):881-885 (1997). Accordingly, there is at least theoretical justification for confining tumor cells within the pelvis for such patients.

FIGS. 1A-1D illustrate operations performed in conjunction with the disclosed embodiments. As shown in FIG. 1A, the tumor bed is injected with magnetic particles. Alternatively, it should be appreciated that tumor-seeking particles may be administered to the patient systemically, e.g., orally, intravenously, etc. (in such a situation the tumor seeking particles may accumulate preferentially in tumor).

As shown in FIG. 1B, the magnetic nanoparticles enter tumor cells and migrate to a sentinel node (optionally identified and/or confirmed using a magnetometer either with or without ultrasound assistance or confirmation).

Subsequently, as shown in FIG. 1C, a source of magnetic field (e.g., magnetic material and/or ferromagnetic material configured to be used as an electromagnet) is placed in regions of tumor and sentinel node prior to a surgical procedure. As shown in FIG. 1D, a surgical procedure may then be performed to remove the primary and/or sentinel node of the tumor. The source of the magnetic field may then be removed after resections.

The main application of the confinement invention is to assist patients undergoing curative surgery. In addition to this main application, confinement of cells may also be a useful strategy in patients who (for various reasons) are poor candidates for curative surgical tumor resection. Clinical data shows continued shedding of tumor cells occurs in patients with unresected gastric cancers (see, S Matsusaka et al, referenced above).

Additionally, many patients with prostate cancer choose to have their tumors treated non-surgically. There is a clear association with the presence of CTC and prostate cancer, and so there is at least theoretical justification for confining tumor cells within the pelvis for such patients. Thus, it should be understood that the tumor confinement methodology could be used in conjunction with application of radiation.

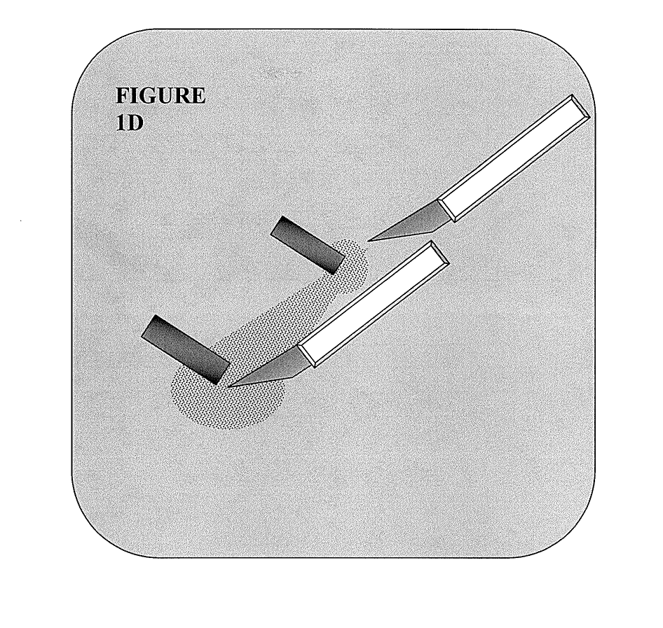

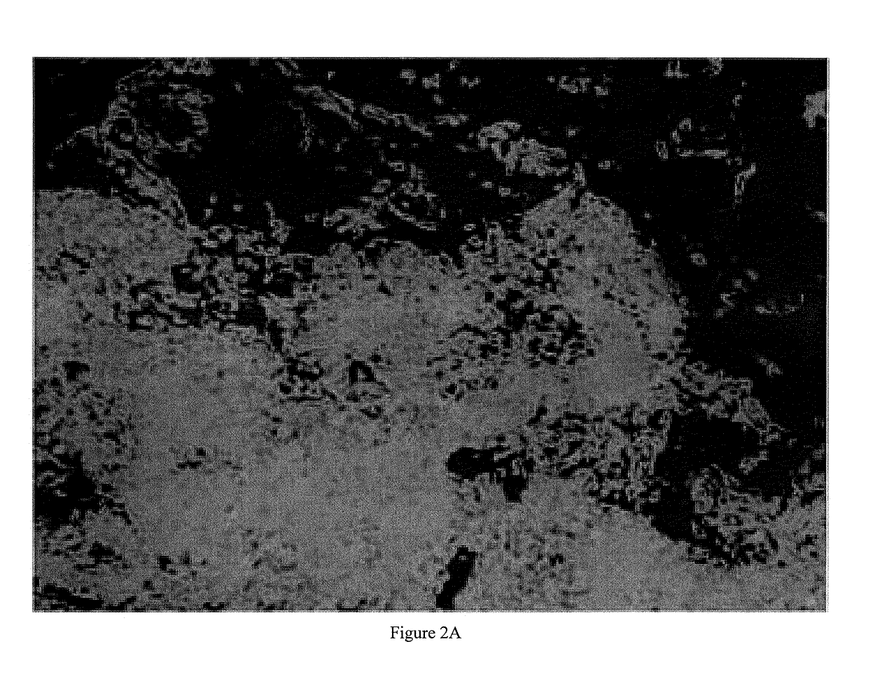

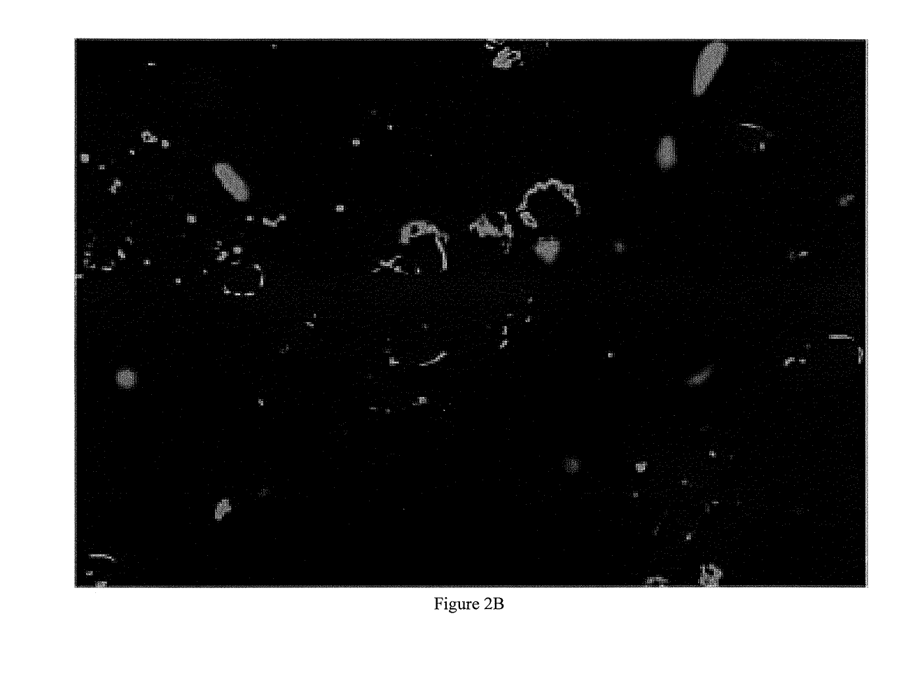

Disclosed embodiments may also be used to increase knowledge of in-vivo behavior of magnetic nanoparticles, based both on animal and human trials (see, A S Lubbe, C Bergemann, H Riess, F Schriever, P Reichardt, K Possinger, M Matthias, B Dorken, F Herrmann, R Gurthler, P Hohenberger, N Haas, R Sohr, B Sander, A-J Lemke, D Ohlendorf, W Huhnt, D Huhn. Clinical Experiences with Magnetic Drug Targeting: A Phase I Study with 4′-Epidoxirubicin in 14 Patients with Advanced Solid Tumors. Cancer Research 56:4686-4693 (1996)), as well as preliminary experiments described in connection with FIG. 2.

The inventors have individually and jointly conducted researched on the behavior of magnetic nanoparticles in vivo, and on optimal design and control of magnets to manipulate magnetic nanoparticles. Additional research has been performed that indicates that a 0.8 Tesla magnet is capable of holding 100-nm magnetic nanoparticles against blood flow in breast cancer and head and neck tumors. See A S Lubbe et al., reference above.

Furthermore, inventors have analyzed the transport of magnetic nanoparticles in vasculature and into surrounding tissue during magnetic targeting, and attained results that correctly predict results in animals and human experiments. Further research has been performed to examine the effect of external magnetic fields on cell cultures that had been loaded with magnetic nanoparticles. As a result of this research, it has been determined that, when the cultures were rinsed, cells near magnets stayed in the dishes, while other cells floated away as shown in FIGS. 2A-B).

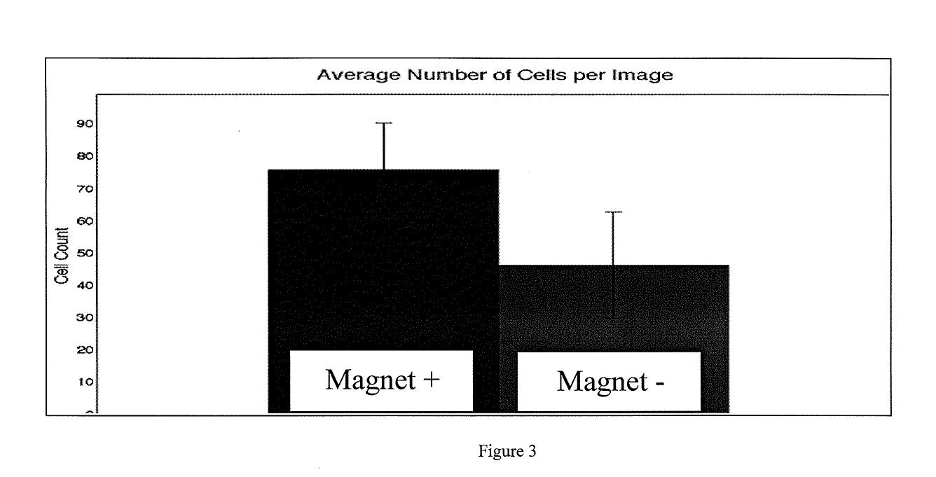

More specifically, FIGS. 2A-B illustrate results of preliminary studies of magnetic confinement in which, MDA-MB-231 cells were cultured onto sixteen cover slips and allowed to grow to confluency. Magnetic ferrofluid (SiMAG-Silanol) was added to media and incubated for one hour. After incubation time, all cover slips were washed aggressively with warm Phosphate Buffered Saline four times. During the washing process, eight cover slips were placed near magnets (see FIG. 2A), and eight were not in the magnetic fields (see FIG. 2B). Cells were stained and images processed quantitatively. The graph illustrated in FIG. 3 illustrates the quantitative analysis of the images to determine the amount of cells that remained as a result of the exposure to the nanoparticles and the magnetic field.

By exploiting the confinement phenomenon illustrated in FIGS. 2A-3, treatment of patients undergoing curative invasive surgery may be supplemented by applying the confinement concept to assist patients undergoing curative surgery; in addition, disclosed embodiments may be utilized in conjunction with the treatment of patients or patient tumors that are poor candidates for curative surgical tumor resection.

As mentioned above, clinical data shows continued shedding of tumor cells occurs in patients with unresected gastric cancers. Thus, the application of magnetic nanoparticles and subsequent application of one or more magnetic fields to minimize or eliminate shedding of cancer cells within the patient's body could have significant benefit at reducing the risk or effect of disseminated metastases.

Utilizing the techniques of the disclosed embodiments may effectively reduce the likelihood for disseminating of cancer cells during surgery. Accordingly, disclosed embodiments may be utilized in a commercial product that may be configured to confine tumor cells to the region of surgery. Additionally, in accordance with disclosed embodiments, CTCs may be detected, correlated with and monitored to determine tumor dissemination. As a result, use of the disclosed embodiments may reduce the number of patients succumbing to metastasis as a result of dissemination or shedding of tumor cells during and after surgery.

It should be understood that the teachings of all the references cited herein are incorporated by reference in their entirety. Therefore, it should be appreciated that techniques disclosed in those references could be used effectively in combination or as part of the processes and techniques disclosed in this specification.

While this invention has been described in conjunction with the specific embodiments outlined above, it is evident that many alternatives, modifications and variations will be apparent to those skilled in the art. Accordingly, the various embodiments of the invention, as set forth above, are intended to be illustrative, not limiting. Various changes may be made without departing from the spirit and scope of the invention.

Additionally, it should be understood that the functionality described in connection with various described components of various invention embodiments may be combined or separated from one another in such a way that the techniques utilized are somewhat different than what is expressly disclosed herein. Moreover, it should be understood that, unless otherwise specified, there is no essential requirement that methodology operations be performed in the illustrated order; therefore, one of ordinary skill in the art would recognize that some operations may be performed in one or more alternative order and/or simultaneously.

Various components of the invention may be provided in alternative combinations operated by, under the control of or on the behalf of various different entities or individuals.

Further, it should be understood that, in accordance with at least one embodiment of the invention, utilized components may be implemented together or separately and there may be one or more of any or all of the disclosed components. Thus, for example, the means for injecting nanoparticles could also provide the ability to trigger entry of those nano-particles into cellular structure or removal of material from cellular structure as disclosed in PCT/US2011/048411, entitled “METHOD AND APPARATUS FOR REMOVING NOXIOUS MATERIALS FROM CELLS.”

Further, components may be either dedicated systems or such functionality may be implemented as virtual systems implemented on general purpose equipment via software implementations.

As a result, it will be apparent for those skilled in the art that the illustrative embodiments described are only examples and that various modifications can be made within the scope of the invention as defined in the appended claims.

What is claimed is:

1. A method for reducing the risk of intra-operative shedding of tumor cells, the method comprising:
introducing magnetic elements into a patient's body; and
exposing the patient to one or more magnetic fields to confine tumor cells to a limited volume in order to prevent the tumor cells from being released elsewhere into the patient's body.

2. The method of claim 1, wherein the magnetic elements are micro or nanoscale objects that are made from or contain magnetically responsive materials such as ferromagnetic, ferrimagnetic, paramagnetic, or diamagnetic materials.

3. The method of claim 1, wherein the tumor cells are isolated from general circulation of the patient and/or lymphatics of the patient.

4. The method of claim 1, wherein the magnetic elements are introduced into the patient's body by injection using a hypodermic needle.

5. The method of claim 1, wherein the magnetic elements are introduced into the patient's body by ingestion.

6. The method of claim 1, wherein the magnetic elements are introduced into the patient's body by intravenous fluid application.

7. The method of claim 1, further comprising the introduction of magnetic elements into tumor cells and then to migrate to a sentinel node of a tumor.

8. The method of claim 1, where the limited volume is a surgical bed.

9. The method of claim 7, wherein the tumor sentinel node is identified and/or confirmed using a magnetometer.

10. The method of claim 7, wherein the tumor sentinel node is identified with ultrasound assistance.

11. The method of claim 1, wherein the exposure to the one or more magnetic fields uses a magnetic field source that includes a permanent-magnet surgical clip.

12. The method of claim 1, wherein the exposure to the one or more magnetic fields uses implantable permanent magnets.

13. The method of claim 1, wherein the exposure to one or more magnetic fields used a permanent or electromagnet held in place by an external assembly, for example a mechanical arm, gentry, or support affixed to the floor, ceiling, wall, table, or other stationary structure external to the patient.

14. The method of claim 1 wherein the exposure to the one or more magnetic fields is performed prior to and during an interventional procedure.

15. A component system for reducing the risk of intra-operative shedding of tumor cells, the device comprising:
a mechanism that introduces magnetic elements into a patient's body; and
at least one permanent or electromagnet configured to confine tumor cells to a limited volume in order to prevent the tumor cells from being released elsewhere into the patient's body.

16. The system of claim 15, wherein the magnetic elements are micro or nanoscale objects that are made from or contain magnetically responsive materials such as ferromagnetic, ferrimagnetic, paramagnetic, or diamagnetic materials.

17. The system of claim 15, wherein the tumor cells are isolated from general circulation of the patient and/or lymphatics of the patient.

18. The system of claim 15, wherein the mechanism for introducing magnetic elements into the patient's body comprises a hypodermic needle.

19. The system of claim 15, wherein the mechanism for introducing magnetic elements into the patient's body utilizes ingestion on the part of the patient's body.

20. The system of claim 15, wherein the mechanism for introducing magnetic elements into the patient's body introduces intravenous fluid into the patient's body.

21. The system of claim 15, further comprising a device configured to introduce the magnetic elements into tumor cells and then to migrate to a sentinel node of a tumor.

22. The system of claim 21, where the limited volume is a surgical bed.

23. The system of claim 21, wherein the tumor sentinel node is identified and/or confirmed using a magnetometer.

24. The system of claim 21, wherein the tumor sentinel node is identified with ultrasound assistance.

25. The system of claim 15, wherein the at least one permanent or electromagnet is held in place by a permanent-magnet surgical clip.

26. The system of claim 15, wherein the at least one permanent magnet or electromagnet is implanted into the patient.

27. The system of claim 15, wherein the at least one permanent or electromagnet is held in place by an external assembly.

28. The system of claim 27, wherein the external assembly includes at least one of a mechanical arm, a gentry, or a support affixed to a floor, ceiling, wall, table, or other stationary structure external to the patient.

29. The system of claim 15, wherein the at lest one permanent or electromagnet is used apply a magnetic force to a portion of the patient's body prior to and during an interventional procedure.
